(19) United States
(12) Patent Application Publication
(10) Pub. No.: US 2002/0183571 A1
(43) Pub. Date: Dec. 5, 2002

(54) Radial reactor loading of a dehydrogenation catalyst

(75) Inventors: David L. Williams, Louisville, KY (US); Andrzej Rokicki, Prospect, KY (US); Dennis J. Smith, Louisville, KY (US); Kyle Mankin, Prospect, KY (US)

Correspondence Address: Scott R. Cox, LYNCH, COX, GILMAN & MAHAN, P.S.C., Suite 2200, 400 West Market St., Louisville, KY, 40202, US

(73) Assignee: Sud-Chemie Inc., Louisville, KY

(21) Appl. No.: 10/041,422

(22) Filed: Jan. 8, 2002

Related U.S. Application Data
(63) Continuation-in-part of application No. 09/923,658, filed on Aug. 7, 2001.
Application No. 09/923,658 is a continuation-in-part of application No. 09/727,036, filed on Nov. 30, 2000.

(51) Int. Cl. C07C 5/373

(52) U.S. Cl. 585/252; 585/330; 585/379; 585/440; 585/616; 585/654; 422/211; 422/213; 422/218; 422/220; 422/222; 422/176; 422/177; 422/181

(57) ABSTRACT

A radial reactor for utilization for catalytic reactions of gaseous or liquid feed streams including an annular catalyst bed, wherein the material contained within the catalyst bed includes an active catalyst material, contained within an outer ring-shaped layer of the catalyst bed, and a generally inert material, contained within an inner ring-shaped layer of the catalyst bed, wherein the generally inert material includes a potassium-containing compound, such as potassium oxide, hydroxide, carbonate or bicarbonate.

### CROSS REFERENCE TO RELATED APPLICATIONS

This application is a continuation-in-part of application Ser. No. 09/923,658, filed Aug. 7, 2001, which was a continuation-in-part of application Ser. No. 09/727,036, filed on Nov. 30, 2000.

### BACKGROUND OF INVENTION

1. Field of Invention.

The field of art to which this invention pertains is to the composition of a dehydrogenation catalyst bed contained in a radial reactor, wherein the dehydrogenation catalyst bed includes a dehydrogenation catalyst material layer and a layer of an inert material containing a potassium compound. More particularly, this invention relates to a dehydrogenation catalyst bed for a radial reactor for dehydrogenation reactions, wherein the catalyst bed is divided into inner and outer ring-shaped layers, wherein catalyst material is placed in the outer layer and inert material containing a potassium compound is placed in the inner layer.

2. Description of related art.

Radial reactors are utilized for a number of different types of catalytic reactions. For example, radial reactors are utilized in ammonia synthesis plants as disclosed in U.S. Pat. Nos. 4,880,603 and 5,250,270.

Various designs for radial reactors and the flow pattern of feed streams within those radial reactors have been disclosed in a number of patents, such as those owned by Ammonia Casale S. A. The reactions that occur in those radial reactors are generally heterogenous synthesis reactions, such as ammonia synthesis or methanol synthesis. The reactors and their catalyst beds are designed to encourage various patterns of flow of the feed stream through the catalyst beds within the radial reactors. These radial reactors are disclosed, for example, in U.S. Pat. Nos. 5,756,048, 5,006,316, 4,963,338, 4,952,375, 4,904,453, 4,755,362, 4,769,220, 4,405,562 and 4,372,920.

A common commercial chemical process where radial reactors are utilized is the dehydrogenation of hydrocarbons. The process for the dehydrogenation of hydrocarbons is well described in the prior art, whereby both acyclic and aromatic hydrocarbons are converted to correspondingly less saturated hydrocarbon products. One of the best known of these dehydrogenation processes is the conversion of alkyl aromatics, particularly ethylbenzene to styrene. In this process ethylbenzene is reacted at an elevated temperature over a dehydrogenation catalyst, such as iron oxide to form styrene. A process for the dehydrogenation of ethylbenzene to styrene and catalysts used for that reaction are disclosed in U.S. Pat. No. 6,096,937. See also U.S. Pat. No. 4,551,571.

The commercial process for the conversion of ethylbenzene to styrene is normally conducted in a series of radial, adiabatic reactors rather than in a single reactor. Radial reactors utilized for the production of styrene are disclosed in U.S. Pat. Nos. 3,475,508, 3,515,763 and 3,918,918. These radial reactors generally are elongated, cylindrical, vertical structures which may be very large, ranging in diameter from 5 to 20 feet (150 to 610 cm) or more and in length from 5 to about 100 feet (150 to 3050 cm) or more. Examples of radial reactor designs are disclosed in the drawings associated with JP 49039971 and 49039972. A process and apparatus for the conversion of ethylbenzene to styrene in a radial reactor are also disclosed in U.S. Pat. Nos. 5,358,698 and 4,039,601.

It has been recognized that a reactor system containing multiple radial reactors may produce a higher degree of conversion of the hydrocarbon and may have greater product yield than is exhibited by use of a single radial reactor. Thus, sometimes three or more radial reactors are arranged in a serial flow orientation with reheat means, which may be located both within and between the reactors, to add heat to the reaction.

Conventional radial reactors contain an inlet located in the center of the radial reactor assembly. Catalysts for the reaction are placed within a bed or beds in that reactor assembly, generally occupying a ring-shaped, vertical space, which is located outside of a central core of the reactor. The feed stream enters the reactor through the inlet and then flows radially outward through the catalyst material contained in the vertical catalyst bed to an open, annular space, which is formed outside of the catalyst bed but within the reactor assembly. Ultimately the feed stream flows to an outlet as shown, for example, in U.S. Pat. No. 3,898,049.

Modifications to this basic design have been considered, such as are disclosed in U.S. Pat. No. 5,358,698. The goal of such modified arrangements is to enhance certain performance and operating characteristics of the catalyst, such as selectivity and activity. “Selectivity” is the ability of a catalyst to selectively produce higher levels of the desired product and lower levels of undesired products. For example, in the conversion of ethylbenzene to styrene, higher selectivity results in higher levels of styrene and lower levels of various by-products. “Activity” is the ability of the catalyst to convert a certain percentage of the ethylbenzene during each pass of the feedstock over the catalyst. Higher activity means a higher percentage of the ethylbenzene is converted.

The conversion of ethylbenzene to styrene is an endothermic reaction which requires the addition of heat to the process to maintain an appropriate level of activity. Accordingly, the rate of flow of the ethylbenzene must be controlled as it passes across the catalyst bed to maintain acceptable selectivity and activity. Thus, the liquid hourly space velocity (LHSV) of the feed stream through the catalyst bed of the reactor must be regulated. The LHSV is generally maintained at a given rate regardless of the thickness of the catalyst bed in the reactor assembly.

Catalyst material loses selectivity and activity over time. Multiple reactors have been used to overcome this problem. One proposed design for such reactors is disclosed in U.S. Pat. No. 6,096,937.

There are two entirely different recognized types of ethylbenzene dehydrogenation reaction. The conventional dehydrogenation reaction (nonoxidative dehydrogenation) occurs over an iron oxide catalyst and requires that oxygen not be present in the feed stream.

The less utilized, second type of dehydrogenation reaction is oxidative dehydrogenation where the feed stream contains ethylbenzene and a source of oxygen. In this second process, the feed stream is passed over an oxidative dehydrogenation catalyst, where an oxidation reaction occurs to generate heat. The reaction is generally as follows:

C₆H₅CH₂CH₃+½O₂→C₆H₅CH=CH₂+H₂O

Oxidative dehydrogenation is exothermic and irreversible. An oxidative dehydrogenation reaction is disclosed for example in U.S. Pat. Nos. 5,510,553 and 4,777,319. This process is also discussed in Kirk-Othmer, Encyclopedia of Chemical Technologies, Volume 22, page 978 (1996).

In addition, UOP has disclosed a process for oxidatively reheating a reaction mixture including ethylbenzene using a radial reactor system containing two oxidation catalyst beds utilized in combination with three conventional dehydrogenation catalyst beds in U.S. Pat. No. 5,043,500. In the process of this patent an oxidation catalyst bed (64) is physically located in a vertically layered bed next to a dehydrogenation catalyst (66) also contained within the reactor. UOP has disclosed the use of one, two or more oxidation catalyst beds. See also Kirk-Othmer, Encyclopedia of Chemical Technologies, Volume 22, pages 978-980 (1996).

Conventional “nonoxidative” dehydrogenation reactions generally use radial reactors but conventionally utilize only a single dehydrogenation catalyst, such as a conventional iron oxide catalyst containing a small amount of potassium and chrome as disclosed, for example, in U.S. Pat. Nos. 2,866,790 and 2,866,791. Various catalysts for nonoxidative dehydrogenation are also disclosed in U.S. Pat. No. 6,191,065, the contents of which are incorporated herein by reference.

Conventional nonoxidative dehydrogenation catalysts used for the conversion of ethylbenzene to styrene in reactors gradually deactivate during normal use, causing a reduction in ethylbenzene conversion. As part of the deactivation process, the catalyst loses potassium. During the dehydrogenation reaction, potassium migrates across the catalyst bed from the inlet side to the outlet side. Thus, the catalyst located closest to the inlet generally exhibits the greatest potassium loss over time.

Several attempts have been made to address the problem of loss of potassium from the dehydrogenation catalyst during a nonoxidative dehydrogenation reaction. For example, patents owned by Raytheon specifically teach methods and apparatus for regenerating and stabilizing dehydrogenation catalysts which have lost potassium during the dehydrogenation reaction. These patents are U.S. Pat. Nos. 5,461,179, 5,686,369, 5,695,724 and 5,739,071. The preferred method comprises the continuous or intermittent introduction of an alkali metal or alkali metal compound to the reactant stream. For example, in one of these processes, a small amount of potassium hydroxide is vaporized continuously into the reaction feed stream. This potassium hydroxide is brought into contact with the catalyst. The patents report that catalyst activity and selectivity were improved by this addition. The process required the addition of from about 0.01 to about 100 parts per million of the alkali metal compound to the feed stream. Additional processes for the addition of the alkali metal compound include addition of the compound in the form of a dry solid powder and the use of a solid lump containing the alkali metal compound placed in the path of the heated reactant feed stream, which solid lump gradually vaporized during processing.

In radial reactors used for ethylbenzene dehydrogenation, the gas feed flows radially from the central core of the reactor assembly through catalyst material contained in a ring-shaped, vertical catalyst bed contained within the radial reactor. However, because many such catalytic reactions, including nonoxidative dehydrogenation of ethylbenzene, are temperature sensitive, the volume of the catalyst material within the catalyst bed that actually catalyzes the feed stream is often limited. For example, in nonoxidative dehydrogenation radial reactors for the dehydrogenation of ethylbenzene, only the first 4 inches (10 cm) to 15 inches (40 cm) or so of thickness of the catalyst material contained in the ring-shaped, vertical layer of the catalyst bed effectively dehydrogenates the ethylbenzene feed stream. This section of the catalyst bed also loses the greatest amount of the potassium during the dehydrogenation reaction. Because the reaction is adiabatic; by the time the ethylbenzene feed stream has passed 18 inches (46 cm) or so through the catalyst bed, the temperature of the feed stream has dropped to such an extent that the activity of the reaction is diminished dramatically or even extinguished. Further, when the temperature of the ethylbenzene feed stream drops as it passes through a thick catalyst bed, a higher percentage of undesired by-products are produced. In addition, the greater the thickness of the catalyst bed, the greater the pressure drop as the feed stream passes through the catalyst bed.

Notwithstanding these reductions in the performance of the catalyst material in the thick catalyst beds contained in large diameter radial reactors, it has become conventional to build shorter reactor assemblies with thicker catalyst beds instead of building taller radial reactors with thinner catalyst beds because of the high cost in building both the support structure for the reactors and the radial reactors themselves. While shorter, thicker radial reactors contain the same overall quantity of catalyst material as taller, thinner radial reactors, the performance of these shorter, thicker reactors is not as efficient as when a taller, but narrower reactor is utilized.

Accordingly, it is an object of this invention to disclose a catalyst composition contained within a catalyst bed of a new or an existing radial reactor assembly which exhibits higher selectivity and/or activity than conventional catalyst compositions.

It is a further object of this invention to disclose a loading of material within a catalyst bed utilized within a new or an existing radial reactor, wherein the material is loaded in an inner and an outer ring-shaped, vertical layer within the catalyst bed, wherein the inner, vertical layer is filled with a generally inert material onto which a potassium-containing compound has been added and the outer, vertical layer is filled with an active catalyst material.

It is a further object of this invention to disclose a loading of material within a catalyst bed for a new or an existing radial reactor utilized for the nonoxidative dehydrogenation of an alkylaromatic feed stream, wherein the material is a combination of a dehydrogenation catalyst, placed within an outer, ring-shaped, vertical layer of the catalyst bed and a generally inert material onto which a potassium-containing compound has been added, placed within an inner, ring-shaped, vertical layer of the catalyst bed of the radial reactor.

It is another object of this invention to disclose a novel process for the nonoxidative dehydrogenation of an alkylaromatic feed stream in a new or an existing radial reactor which includes passing the alkylaromatic feed stream through a catalyst bed in the radial reactor, wherein the catalyst bed contains a nonoxidative dehydrogenation catalyst placed within an outer, ring-shaped, vertical layer of the catalyst bed and a generally inert material onto which a potassium-containing compound has been added, placed within an inner, ring-shaped, vertical layer of the catalyst bed.

These and other objects can be obtained by the disclosed composition of the materials contained in catalyst beds of new or existing radial reactors and processes for the loading and utilization of those radial reactors.

### SUMMARY OF THE INVENTION

This invention is directed to a composition of material loaded into a catalyst bed of a radial reactor for catalytic reactions of gaseous or liquid feed streams, particularly for the dehydrogenation of alkyl aromatics, wherein the radial reactor comprises a conventional radial reactor assembly containing an annular, ring-shaped, vertical catalyst bed, and wherein the material loaded within the catalyst bed comprises an active catalyst material, contained within a first ring-shaped, vertical layer of the catalyst bed, and a generally inert material onto which a potassium-containing compound has been added, contained within a second, ring-shaped, vertical layer of the catalyst bed. The catalyst material is contained within an outer ring-shaped layer of the catalyst bed and the generally inert material onto which a potassium-containing compound has been added is contained within an inner ring-shaped layer of the catalyst bed. By “generally inert material with an added potassium-containing compound,” is meant a material that will not adversely impact the physical or chemical characteristics of the catalyst material during reaction yet replace a portion or all of the potassium that is lost from the active catalyst material during catalytic activity. Preferably the thickness of the layer that holds the catalyst material is from about 4 inches (10 cm) to about 48 inches (120 cm), more preferably from about 6 inches (15 cm) to about 36 inches (90 cm), most preferably about 18 inches (46 cm) to 24 inches (60 cm).

In a preferred embodiment, the generally inert material comprises a material onto which a potassium-containing compound has been added and which does not interfere with the desired catalytic reaction that occurs within the radial reactor. Also, preferably, the use of the generally inert material in the inner, ring-shaped layer, results in no additional pressure drop of the feed stream, and most preferably, it results in a reduced pressure drop of the feed stream in comparison with the pressure drop that occurs if the catalyst bed contained only active catalyst material. In a further preferred embodiment the generally inert material comprises an alpha alumina or ceramic material onto which a potassium-containing compound has been added. The size of this inert material onto which the potassium-containing compound has been added is preferably similar to that of the active catalyst material. Different sized or shaped materials can be utilized as the generally inert material, such as a monolithic structure, as long as there is not significant movement of either the catalyst or the inert material onto which a potassium-containing compound has been added within the catalyst bed after loading.

This invention is also directed to a process for the nonoxidative dehydrogenation of an alkylaromatic feed stream comprising passing the alkylaromatic feed stream through a radial reactor containing a catalyst bed, wherein the catalyst bed comprises an inner, ring-shaped layer and an outer, ring-shaped layer and wherein nonoxidative dehydrogenation catalyst material is loaded within the outer layer and a generally inert material onto which is placed a potassium-containing compound is loaded within the inner layer.

### BRIEF DESCRIPTION OF THE DRAWING

FIG. 1 is a schematic view of a radial reactor of the invention.

FIG. 2 is a top view of the radial reactor of FIG. 1.

FIG. 3 is a cut-away perspective view of the catalyst bed of the radial reactor of FIG. 1.

### DETAILED DESCRIPTION OF THE INVENTION

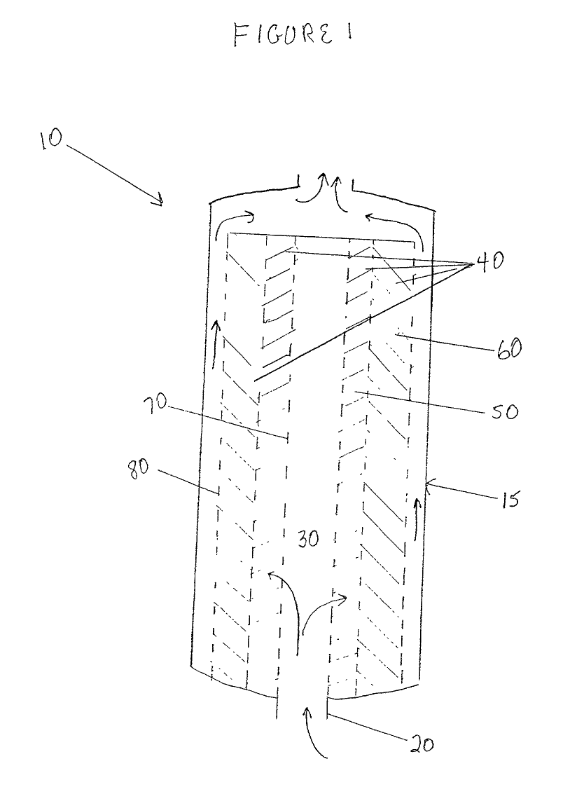

The invention is a composition for material loaded within a catalyst bed (40) within a radial reactor (10) utilized for catalytic reactions of gaseous or liquid feed streams, which reactor (10) includes a conventional radial reactor assembly (15), which contains a vertical, annular catalyst bed (40), wherein the catalyst bed is loaded with an active catalyst material (60), contained within a first ring-shaped layer of the catalyst bed (40), and a generally inert material (50) onto which a potassium-containing compound has been added contained within a second ring-shaped layer of the catalyst bed (40). Preferably, the first and second ring-shaped layers are in a vertical position in the radial reactor in relation to the facility in which the radial reactor is located as shown in FIG. 1.

Referring to FIG. 1, which is a cross-sectional schematic drawing showing one embodiment of the radial reactor (10) of the invention, a feed stream is introduced through an inlet pipe (20) into the center (30) of the radial reactor (10). When the reaction is the preferred nonoxidative dehydrogenation reaction, the hydrocarbon feed stream comprises an alkylaromatic material, preferably ethylbenzene. In this reaction the feed stream is heated by mixing it with super heated steam. The heated feed stream enters the inlet pipe (20) of the radial reactor (10) and is radially distributed through the catalyst bed (40) as shown by the arrows in FIG. 1.

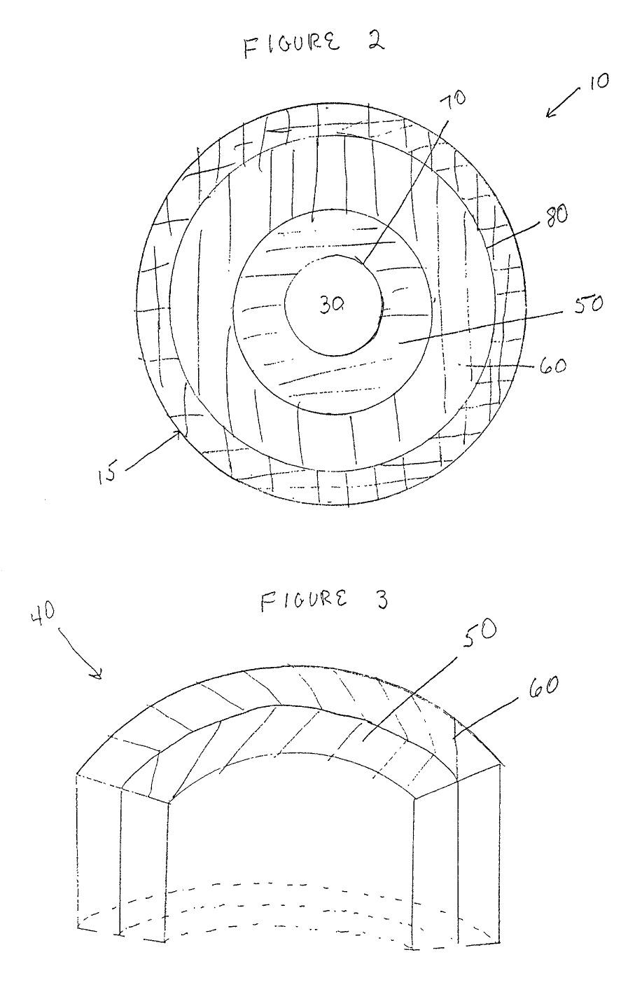

The catalyst bed (40) of the invention is comprised of two or more layers of material, preferably arranged in ring-shaped, vertical layers of material loaded in the reactor bed (40) as shown in FIGS. 1, 2 and 3. (While only a pair of vertical layers are shown in FIGS. 1, 2 and 3, three or more layers are within the scope of the invention. Other arrangements of the layers of material are also within the scope of the invention.) When two (2) layers of material are loaded within the catalyst bed (40), the two (2) layers preferably comprise a layer of an active catalyst material (60) and a layer of a generally inert material (50) onto which a potassium-containing compound has been added. For example, when the reaction is the nonoxidative dehydrogenation of ethylbenzene, one layer of the catalyst bed is comprised of an active catalyst (60) for that reaction and the second layer is comprised of a generally inert material (50), which does not interfere with the nonoxidative dehydrogenation reaction and which contains a potassium-containing compound.

Each of the materials are preferably arranged in a separate vertical, annular layer within the radial reactor (10) as shown in FIG. 1. Although formal separation of the two vertical layers of material can be effected by use of a device such as a screen, such as is shown in U.S. Pat. No. 5,043,500, in a preferred embodiment the two separate layers of material (50, 60) are in intimate contact with each other within the radial reactor (10). In this preferred arrangement some mixing of the catalyst material with the generally inert material may occur at the boundary area between the different materials, although substantial mixing is not preferred.

The materials are retained in place within the catalyst bed (40) of the radial reactor (10) by use of an inner screen (70) and an outer screen (80), as shown in FIG. 1.

In a preferred embodiment, the catalyst bed (40) contains at least two vertical layers (50, 60) of material, wherein at least one of the layers of material is the active catalyst material (60) and at least one of the layers is the inert material (50) onto which a potassium-containing compound has been added. Many different combinations of active catalysts and generally inert materials containing a potassium-containing compound are within the scope of the invention. For example, in a preferred embodiment for the non-oxidative dehydrogenation of or alkyl aromatic feed stream, a nonoxidative dehydrogenation catalyst, which has high activity is placed in the outer layer (60) of the catalyst bed (40) and a generally inert material, such as a ceramic or alpha alumina material onto which a potassium-containing compound has been added, is placed within the inner layer (50) within the radial reactor (10).

By placing the active catalyst material in the outer ring-shaped, vertical layer (60) of the catalyst bed (40), there is an increase in the overall initial surface area of the catalyst material that is exposed to the hot, preheated feed stream as the feed stream begins passage through the catalyst bed (40) as shown in FIG. 3. There is an increase in the overall initial surface area of the catalyst layer because it is a further distance from the center (30) of the radial reactor (10) as shown in FIGS. 1, 2 and 3, thus presenting a greater vertical surface of catalyst material for reaction with the hot, preheated feed stream. Because the generally inert material containing a potassium-containing compound does not interfere with the reaction and because a greater surface area of the catalyst material is exposed to the feed stream, there is an increase in performance of the overall catalyst bed (40) over a conventional catalyst bed, wherein the inner ring-shaped, vertical layer (50) of the catalyst bed (40) is also filled with catalytic material.

This arrangement of material in a radial reactor (10) results in a greater volume of catalytic material that is “effectively” utilized in the catalyst bed (40). In most reactions, the “effective” portion of the catalyst bed (40) that is utilized is only the first 4 inches (10 cm) to about 15 inches (40 cm) or so of the thickness of the catalyst bed (40). Because the design of the catalyst loading of the catalyst bed of the invention has shifted the effective volume of catalytic material further outward within the catalyst bed (40), as shown, for example, in FIG. 3, there is a greater overall volume of catalyst material contained within the first 4 to 15 inches (10-40 cm) of the active catalyst material using the design of the catalyst bed of the invention than would be present in the first 4 to 15 inches (10-40 cm) of a catalyst bed that did not contain inert material containing a potassium-containing compound within an inner ring-shaped, vertical layer (50) of the catalyst bed (40).

There are other improvements which result from this combination of a generally inert material containing a potassium-containing compound utilized with a catalytic material. For example, because the inert material containing a potassium-containing compound preferably has greater physical stability than that of the catalytic material which it replaces, there is a reduction in both the initial pressure drop and the increase in pressure drop with aging of catalyst as the feed stream passes radially through the catalyst bed (40). The liquid hourly space velocity (LHSV) is also increased.

One of the results of the use of this combination of a generally inert material containing a potassium-containing compound and a catalytic material is a continuous addition of potassium to the catalytic material to replace partially or totally the potassium lost by the catalyst material during the non-oxidative dehydrogenation reaction. Conventional catalytic materials utilized for a dehydrogenation reaction contain from about 1 to about 20 percent potassium. During a non-oxidative catalytic dehydrogenation reaction, the highest temperature of reaction occurs near the edge of the catalytic material that is closest to the inlet (20) of the feed stream. Although selectivity and activity of the catalyst material is highest at this location, potassium contained in this catalytic material also begins to migrate away from this catalytic material quickest at this location, resulting in a reduction of the overall performance of the catalytic material.

In contrast, this arrangement of a generally inert material containing a potassium-containing compound and a catalytic material of the invention permits the potassium contained within the generally inert material to migrate from that inert material to the catalytic material, replacing potassium which has been lost from the catalytic material during catalytic activity. This replacement of potassium allows the catalytic material to retain its catalytic activity for a period of time greater than is experienced by catalytic material without the addition of a potassium-containing compound.

This arrangement also reduces unwanted byproducts that are often produced by the reaction of catalytic material in a catalyst bed (40) which is very thick. For example, when a radial reactor (10) is utilized for the nonoxidative dehydrogenation of ethylbenzene to styrene, the temperature within the catalyst bed (40) drops dramatically as the feed stream passes radially through the catalyst material within the catalyst bed (40). Because other types of reactions are preferentially catalyzed by the catalyst material at lower temperatures, there is an increase in the amount of unwanted by-products that are produced the further the feed stream passes through the catalyst bed (40). By reducing the overall thickness of the active catalyst material within the catalyst bed (40) and by replacing the active catalyst material with the generally inert material containing a potassium-containing compound, there is a reduction in the overall by-products produced. This reduction in byproducts occurs because there is a reduction in the quantity of the catalyst material that is exposed to the feed stream after the main reaction has occurred. Because the generally inert material containing a potassium-containing compound does not react with the components of the feed stream and because the radial reactor (10) is a closed system, there is no drop in the temperature of the feed stream during its passage through the inert material containing a potassium-containing compound. By reducing the overall thickness of the catalyst bed (40), there is less catalyst that is exposed to the feed stream at a lower temperature than with conventional catalyst beds, thus resulting in a reduction in unwanted byproducts.

The thickness of the active catalyst material layer of the catalyst bed (40) of the invention may vary depending upon the type of reaction that is being catalyzed by the catalyst material. In one preferred embodiment when the catalyst material is utilized for the nonoxidative dehydrogenation of ethylbenzene to styrene, the effective thickness of the catalytic material is from about 4 inches (10 cm) to about 48 inches (120 cm), preferably from about 6 inches (15 cm) to about 36 inches (90 cm), most preferably from about 18 inches (45 cm) to about 24 inches (60 cm). When the thickness of the catalyst bed (40) in the radial reactor (10) is greater than the desired effective thickness of catalyst material for the catalytic reaction, the remaining space within the catalyst bed (40) is filled with the generally inert material to which a potassium-containing compound has been added. The thickness of the ring-shaped, vertical layer (50) of the generally inert material containing a potassium-containing compound will thus vary depending upon the overall thickness of the catalyst bed (40) within the radial reactor (10) and depending on the type of reaction being catalyzed by the catalyst material. Conventional catalyst beds may vary in thickness from as thin as 18 inches (45 cm) or so to 4 feet (120 cm) or more. Thus, the amount of the generally inert material containing a potassium-containing compound varies depending upon the amount of catalytic material necessary for an effective reaction. Regardless, any reduction in the amount of the catalyst to the optimum range of four (4) inches (10 cm) to about forty-eight (48) inches (120 cm) is helpful and results in an enhancement in the activity of the catalyst within the catalyst bed.

In one preferred embodiment for the dehydrogenation of ethylbenzene as shown in FIG. 1, the dehydrogenation catalyst contained in the outer, ring-shaped, vertical layer (60) is any conventional commercial or proprietary dehydrogenation catalyst, such as Styromax® catalyst produced by Süd-Chemie Inc., which catalyst material is comprised of iron oxide and potassium oxide. In a particularly preferred embodiment the nonoxidative dehydrogenation catalyst material contained in the outer, ring-shaped, vertical layer (60) is selected from the catalysts disclosed in U.S. Pat. Nos. 6,242,379, 6,191,065 and 6,177,602, which are incorporated herein by reference.

Two or more nonoxidative dehydrogenation catalysts may be utilized together within the active catalyst material layer of the radial reactor (10), each forming a different vertical layer as long as the overall thickness of the layer of the active catalyst material does not dramatically reduce the overall performance of the radial reactor (10). When more than one layer of nonoxidative dehydrogenation catalysts is utilized, preferably at least one of the catalysts has a different performance and/or operating characteristic than at least one of the other catalysts. Different layers of the same catalysts may also be sandwiched around a catalyst with different operating or performance characteristics, depending upon the overall performance or operating characteristics that are desired.

The generally inert material containing a potassium-containing compound that is utilized within the catalyst bed (40) is preferably any material which does not adversely interfere with the catalytic reaction of the catalyst material contained in the catalyst bed (40). This generally inert material containing a potassium-containing compound also should not react with the components of the feed stream to produce unwanted byproducts. Further, the generally inert material containing a potassium-containing compound is preferably formed in a shape which limits the overall pressure drop through the catalyst bed (40). Further, the generally inert material containing a potassium-containing compound should have adequate crush strength. The crush strength is preferably the same as or greater than that of the active catalyst material. The generally inert material containing a potassium-containing compound is also preferably formed in a similar size and shape to the catalytic material for loading purposes, but can have larger or more numerous openings passing through the individual particles of the inert material containing a potassium-containing compound to reduce the overall pressure drop.

When the reaction is a nonoxidative dehydrogenation reaction, the generally inert material is preferably an inert material with a surface area from about 0.1 to about 50 m^{2/}g, preferably from about 1 to about 20 m²/g, such as an alpha alumina or a ceramic material including ceramic monoliths. The generally inert material that is chosen should be one that can receive and adsorb an appropriate amount of the potassium-containing compound. The amount of the potassium-containing compound that is added to the inert material should be sufficient to replace substantially any potassium that is lost from the dehydrogenation catalyst during conventional processing of the alkylaromatic feed stream over the conventional life of the catalyst bed. The amount of the potassium-containing compound that is added to the inert base material depends on the composition of the generally inert material and may vary, sometimes dramatically. However, in a preferred embodiment, the amount of the potassium-containing component that is added to the inert material should comprise, after addition, at least about 0.1 percent, preferably from about 1 to about 40 percent and most preferably from about 5 to about 20 percent of the inert material, by weight.

The potassium-containing compounds that can be added to the inert base material include potassium oxide, potassium hydroxide, potassium carbonate and potassium carbonate or other similar potassium compounds and combination thereof. Potassium chloride should not be utilized as chloride ions may adversely affect the catalyst.

In one preferred embodiment of a nonoxidative dehydrogenation process of the invention as shown in FIGS. 1, 2 or 3, nonoxidative dehydrogenation catalysts are loaded into the outer, vertical layer (60) and generally inert materials containing the potassium-containing compound are loaded in the inner, vertical layer (50) of the catalyst bed (40) of the radial reactor (10), forming separate, vertical layers within the radial reactor (10). In this preferred embodiment, the outer layer (60) of catalyst material is at least four (4) inches (10 cm) thick, preferably from about four (4) inches (10 cm) to about forty-eight (48) inches (120 cm) thick and most preferably from about six (6) inches (15 cm) to about thirty-six (36) inches (90 cm) thick. The remaining material (50) in the catalyst bed (40) is the generally inert material containing the potassium-containing compound, preferably an alpha alumina, ceramic material or a monolithic structure containing potassium oxide, hydroxide, carbonate or bicarbonate. It is not mandatory that all of the generally inert material contain a potassium-containing compound. However, sufficient potassium-containing compound should be added to the inert material so that it can replace a significant portion of the anticipated lost potassium material that was present in the active catalyst component.

Because the generally inert material containing a potassium-containing compound is placed closest to the source of heat, potassium compounds are encouraged to migrate from the inert material to the active catalyst material. Thus, an amount of potassium-containing compound in excess of that required to replace all of the potassium material contained in the active catalyst material in the radial reactor may be helpful in assisting the retention of activity and selectivity of the dehydrogenation catalyst.

After the nonoxidative dehydrogenation catalyst and the generally inert material containing the potassium-containing compound are loaded into the catalyst bed (40) of the radial reactor (10), the feed stream, preferably an alkylaromatic and steam, is then passed through the radial reactor (10).

By using this composition of materials for the 5 catalyst bed (40), significant performance advantages are achieved in comparison to the performance of conventional radial reactors with catalyst beds with a thickness greater than about 18 to 48 inches (45-120 cm) which are loaded with only active catalyst material. Using the inventive design wherein the generally inert material contains a potassium-containing compound, the life of the catalytic material is lengthened and the overall selectivity and activity of the catalyst material is enhanced for an extended period of time. In addition, in the inventive design, the catalyst material in the outer ring-shaped, vertical layer (60) presents a higher surface area for reaction with the feed stream than if only catalyst material is utilized within the catalyst bed (40) because the overall surface area of the catalyst material portion of the catalyst bed (40) is greater the further one moves radially outward from the center (30) of the radial reactor (10) as shown in FIG. 3. In addition, because the volume of the catalyst material which is exposed to the feed stream at the proper operating parameters is optimized, there is a greater “effective” utilization of the catalyst material, thus resulting in higher performance of the catalyst material within the catalyst bed (40). Further, by choosing the right size, shape and strength of the inert material containing a potassium-containing compound, there is no increase in pressure drop and preferably there is a reduction in the pressure drop as the feed stream passes through the catalyst bed (40). In addition, the effective LHSV is increased. Less unwanted byproducts are also produced because the catalyst material is more effectively utilized. Finally, depending upon the relative cost of the catalyst material versus the generally inert material containing the potassium-containing compound, there may be a reduction in the overall cost of a catalyst load within the catalyst bed of the radial reactor.

The principles, preferred embodiments and modes of the operation of the present invention have been described in the foregoing specification. The invention which is protected herein, however, is not to be construed as limited to the particular forms disclosed as these are to be regarded as illustrative rather than restrictive. Variations and changes may be made by those skilled in the art without departing from the spirit of the invention.

1. A radial reactor for catalytic reaction of gaseous or liquid feed streams comprising
a radial reactor assembly containing an annular catalyst bed, and
a ring-shaped layer of material contained within the catalyst bed, wherein the material comprises an active catalyst material contained within a first ring-shaped layer of the catalyst bed and a generally inert material contained within a second ring-shaped layer of the catalyst bed, wherein a potassium-containing compound has been added to the inert material.

2. The radial reactor of claim 1 wherein the active catalyst material is contained within an outer ring-shaped layer of the catalyst bed and the generally inert material with added potassium-containing compound is contained within an inner ring-shaped layer of the catalyst bed.

3. The radial reactor of claim 1 wherein the generally inert material comprises an alpha alumina, ceramic material or a monolithic structure and a potassium-containing compound.

4. The radial reactor of claim 3 wherein the potassium-containing compound is selected from the group consisting of potassium oxide, potassium hydroxide, potassium carbonate, and potassium bicarbonate.

5. The radial reactor of claim 1 wherein the overall thickness of the ring-shaped layer of material contained within the radial reactor is at least about 18 inches (45 cm).

6. The radial reactor of claim 1 wherein the overall thickness of the ring-shaped layer of material is from about 18 inches (45 cm) to about 48 inches (120 cm).

7. The radial reactor of claim 1 wherein the active catalyst material comprises a plurality of active catalyst products, at least two of which catalyst products have different performance characteristics.

8. The radial reactor of claim 1 wherein the added potassium-containing compound comprises at least about 0.1 percent of the inert material, by weight.

9. The radial reactor of claim 1 wherein the potassium-containing compound comprises from about 5 to about 20 percent of the inert material, by weight.

10. A radial reactor for nonoxidative dehydrogenation of an alkylaromatic feed stream comprising
a radial reactor assembly containing an annular catalyst bed and
a ring-shaped layer of material contained within the catalyst bed, wherein the material comprises a nonoxidative dehydrogenation catalyst contained within an outer, ring-shaped layer of the catalytic material and a generally inert material contained within an inner ring-shaped layer of the catalyst bed, wherein a potassium-containing compound has been added to the inert material.

11. The radial reactor of claim 10 wherein the inert material comprises an alpha alumina or a ceramic material and a potassium compound.

12. The radial reactor of claim 10 wherein the potassium-containing compound comprises at least about 0.1 percent of the inert material, by weight.

13. The radial reactor of claim 10 wherein the potassium-containing compound comprises from about 5 to about 20 percent of the inert material, by weight.

14. A process for the nonoxidative dehydrogenation of an alkylaromatic feed stream comprising
passing an alkylaromatic feed stream through a radial reactor, wherein the radial reactor comprises
a radial reactor assembly containing an annular catalyst bed, and
a ring-shaped layer of material contained within the catalyst bed, wherein the material comprises an alkylaromatic catalyst material contained within an outer ring-shaped layer of the catalyst bed and a generally inert material contained within an inner ring-shaped layer of the catalyst bed, wherein a potassium-containing compound has been added to the inert material.

15. The process of claim 14 wherein the inert material comprises an alpha alumina or a ceramic material and a potassium-containing compound.

16. The radial reactor of claim 14 wherein the potassium-containing compound comprises at least about 0.1 percent of the inert material.

17. The radial reactor of claim 14 wherein the potassium-containing compound comprises from about 5 to about 20 percent of the inert material.

18. The process of claim 14 wherein the annular catalyst bed is from about 18 inches (45 cm) to about 48 inches (120 cm) in thickness.

19. The process of claim 14 wherein the alkylaromatic catalyst material comprises a plurality of alkylaromatic catalyst products, at least two of which products have different performance characteristics.

20. A process for the nonoxidative dehydrogenation of ethylbenzene comprising
passing an ethylbenzene feed stream through a radial reactor, wherein the radial reactor comprises
a radial reactor assembly containing an annular catalyst bed, and
a ring-shaped layer of material contained within the catalyst bed, wherein the material comprises an ethylbenzene dehydrogenation catalyst contained within an outer, ring-shaped layer of the catalyst bed and a generally inert material contained within an inner, ring-shaped layer of the catalyst bed, wherein a potassium-containing compound has been added to the inert material.

21. The process of claim 20 wherein the inert material comprises an alpha alumina, a ceramic material or a monolithic structure and a potassium-containing compound.

22. The radial reactor of claim 20 wherein the potassium-containing compound comprises at least about 0.1 percent of the inert material.

23. The radial reactor of claim 20 wherein the potassium compound comprises from about 5 to about 20 percent of the inert material.
